(19) United States
(12) Patent Application Publication
(10) Pub. No.: US 2004/0037873 A1
(43) Pub. Date: Feb. 26, 2004

(54) Norethindrone sustained release formulations and methods associated therewith

(75) Inventors: Angela Anigbogu, Salt Lake City, UT (US); Ana Ruiz, Salt Lake City, UT (US); Danyi Quan, Salt Lake City, UT (US)

Correspondence Address: M. Wayne Western, THORPE NORTH & WESTERN, LLP, P.O. Box 1219, Sandy, UT, 84091-1219, US

(73) Assignee: WATSON PHARMACEUTICALS, INC.

(21) Appl. No.: 10/449,861

(22) Filed: May 30, 2003

Related U.S. Application Data
(60) Provisional application No. 60/383,790, filed on May 30, 2002.

(51) Int. Cl. A61F 13/00

(52) U.S. Cl. 424/449

(57) ABSTRACT

Sustained delivery formulations of norethindrone are disclosed and described. In one aspect, the formulation may be a transdermal formulation that includes both norethindrone and norethindrone acetate. In another aspect, the formulation may further include a penetration enhancer. Coadministration of norethindrone and norethindrone acetate has been found to provide a number of advantages, such as achievement of peak norethindrone serum levels substantially within 24 hours after initiation of administration.

### PRIORITY DATA

This application claims priority to U.S. Provisional Patent Application Serial No. 60/383,790, filed on May 30, 2002, which is incorporated herein by reference.

### FIELD OF THE INVENTION

The present invention relates to sustained release transdermal formulations of norethindrone and methods associated therewith. Accordingly, this invention covers the fields of pharmaceutical sciences, medicine and other health sciences.

### BACKGROUND OF THE INVENTION

Female hormones, such as estrogens and progestins have been indicated for a number of medicinal uses, such as hormone replacement therapy (HRT), and contraceptives for women. Both oral and transdermal dosage forms containing estrogens or progestins are well known, and often both are administered together in a single formulation. Due to the strict nature and perpetual duration of HRT and contraception, transdermal formulations are an attractive alternative to instant release oral dosage forms. However, because the skin is a formidable barrier for most drugs, transdermal administration typically requires a greater amount of time to attain significant onset of action, and provide the desired therapeutic effect.

One specific progestin that has received much attention is norethindrone (NE) and its prodrug norethindrone acetate (NEA). Both compounds have been transdermally administered as part of a number of specific formulations. Examples of such formulations are contained in U.S. Pat. Nos. 5,211,952, 5,252,334, 5,422,119, 5,770,219, 5,783,208, 5,980,932, 6,149,935, and 6,465,004, each of which is incorporated herein by reference. U.S. Pat. Nos. 5,770,219, and 6,149,935 to Chiang et al. (collectively referred to as “Chiang”), disclose “[m]onolith matrix systems * * * based on estradiol, norethindrone, norethindrone acetate, and levonorgesterol”. Further, in order to minimize or remove the need for traditional penetration enhancers, Chiang provides “a matrix layer [with dispersed drug] made up of a pressure-sensitive vinyl acetate-acrylate copolymer”. Flux rates achieved for transdermal devices containing norethindrone acetate and norethindrone respectively, as well as the prescribed matrix system are separately evaluated in the examples of each reference.

A drawback to the Chiang technology is that Chiang fails to attain a maximum drug serum concentration in a rapid manner. Like many other transdermal systems, and as illustrated by FIG. 3, maximum skin flux for NEA is attained only after the second day of administration. As a result, the daily dosage received during the first day of administration may be inadequate to attain a needed effect. For example due to the strict dosing requirements posed by a contraception regimen, failure to attain required norethindrone blood levels during the first day of administration from a transdermal patch may be tantamount to skipping a day of administration in an oral dosage regimen and may increase the risk of pregnancy.

Therefore, transdermal formulations of norethindrone that provide more rapid attainment of norethindrone serum levels continue to be sought through ongoing research and development efforts.

### SUMMARY OF THE INVENTION

Accordingly, the present invention provides transdermal compositions and methods for the administration of female hormones to a subject. In one aspect, such a transdermal composition may include a pharmaceutically acceptable transdermal carrier, and a therapeutically effective amount of norethindrone and norethindrone acetate in the carrier. In another aspect, the transdermal composition may further include a therapeutically effective amount of an estrogenic steroid.

The norethindrone and norethindrone acetate elements may be included in the transdermal composition of the present invention in a number of varying individual concentrations and ratios to one another. However, in one aspect, the norethindrone and norethindrone acetate may be present in a weight ratio of from about 1:1 to about 1:25. In another aspect, the ratio may be from about 1:2 to about 1:8.

The estrogenic steroid may also vary in type and amount. In one aspect, the estrogenic steroid may be an estradiol. In another aspect, the estradiol may be ethinyl estradiol. In a further aspect, the amount of estrogenic steroid may be sufficient to provide a therapeutic effect that is equivalent to an effect produced by ethinyl estradiol administered from an adhesive matrix patch in an amount of from about to about 25 to 45 ug/cm².

In addition to the norethindrone, norethindrone acetate, and optional estrogenic steroid active agents, the transdermal compositions of the present invention may optionally include one or more penetration enhancers. In one aspect, the penetration enhancer may be selected from the group consisting of: lauryl-type enhancers, polyol-type enhancers, and mixtures thereof. In another aspect, the enhancer may be a lauryl-type enhancer selected from the group consisting of: lauryl alcohol, 1-lauryl-2-pyrrolidone, and mixtures thereof. In yet another aspect, the enhancer may be a mixture of lauryl alcohol and 1-lauryl-2-pyrrolidone. In a further aspect, the enhancer may be a polyol-type enhancer. In an additional aspect, the polyol-type enhancer may be dipropylene glycerol.

While the amount of penetration enhancer used may vary depending on a number of criteria, such as the type of enhancer selected, the material of the carrier, etc., in one aspect, the enhancer amount may be from about 0.01% w/w to about 50% w/w of the transdermal composition. In another aspect, the enhancer amount may be from about 3% w/w to about 8% w/w of the transdermal composition.

A number of pharmaceutically acceptable carriers are known to those of ordinary skill in the art and may be used in connection with the present invention. However, in one aspect, the carrier may be a polymeric adhesive matrix. In another aspect, the polymeric adhesive may be an acrylic pressure sensitive adhesive.

The transdermal composition of the present invention may be configured to provide a number of specific results which present an advantage over compositions of the prior art. For example, in one aspect, the transdermal compositions recited herein may provide a subject, to which the composition is administered, with a maximum norethindrone serum concentration within about 24 hours after initiation of administration. In another aspect, the compositions may provide a subject with a substantially higher norethindrone serum concentration than an equivalent dosage of a transdermal composition containing either norethindrone or norethindrone acetate alone. Such synergistic results are unexpected and extremely advantageous.

The present invention additionally encompasses various methods of making and use associated with the transdermal compositions disclosed herein. In one aspect, the present invention includes a method of transdermally providing a subject with a maximum norethindrone serum concentration within about 24 hours after initiation of transdermal administration by coadministering norethindrone and norethindrone acetate to the skin of the subject. In another aspect, a method is provided for exceeding a norethindrone serum concentration achieved in a subject by transdermal delivery of either norethindrone alone or norethindrone acetate alone, by administering to the skin of a subject a combined amount of norethindrone and norethindrone acetate that is equivalent to an amount of either the norethindrone or norethindrone acetate alone. Additionally, the present invention provides a method of enhancing norethindrone and norethindrone acetate permeation through the skin of a subject by coadministering the norethindrone and norethindrone acetate with a permeation enhancer selected from the group consisting of: lauryl alcohol, 1-lauryl-2-pyrrolidone, dipropylene glycerol, and mixtures thereof to the skin

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION

A. Definitions

In describing and claiming the present invention, the following terminology will be used in accordance with the definitions set forth below.

The singular forms “a,” “an,” and, “the” include plural referents unless the context clearly dictates otherwise. Thus, for example, reference to “an adhesive” includes reference to one or more of such adhesives, and reference to “an excipient” includes reference to one or more of such excipients.

As used herein, “estrogen” and “estrogenic hormone” may be used interchangeably, and refer to any substance, natural or synthetic, that exerts a biological or pharmacological action primarily by binding to estrogen receptors. Examples include but are not limited to: 17-β-estradiol, 17-α-estradiol, estriol, estrone, and phytoestrogens. These estrogens may be derivatized or modified to form, for example, conjugated equine estrogens, esterified estrogens, ethinyl estradiol, etc. Examples of esterified estrogens include but are not limited to: estradiol-3,17-diacetate, estradiol-3-acetate, estradiol-17-acetate, estradiol-3,17-divalerate, estradiol-3-valerate, estradiol-17-valerate. The estrogens may also be present as salts, (e.g., as sodium estrogen sulfate), isomers, or prodrugs.

As used herein in, “norethindrone,” or “NE” refers to a compound having the general chemical structure:

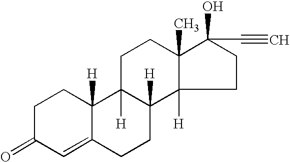

Norethindrone is well known in the art, and is listed as monograph 6790 on pg. 1149 of the Merck Index (12^{th} ed. 1996), which is incorporated herein by reference.

As used herein, “norethindrone acetate,” or “NEA” refers to a compound having the general chemical structure:

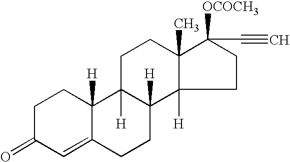

Norethindrone acetate is well known in the art as an ester-type prodrug of norethindrone and is described on pg. 1096 of Remington: The Science and Practice of Pharmacy (19^{th} ed. 1995), which is incorporated herein by reference.

As used herein, “subject” refers to a mammal that may benefit from the administration of a drug composition or method of this invention. Examples of subjects include humans, especially females, and may also include other animals such as horses, pigs, cattle, dogs, cats, rabbits, and aquatic mammals.

As used herein, the terms “formulation” and “composition” are used interchangeably. The terms “drug,” “pharmaceutical,” “active agent,” and “bioactive agent” are also used interchangeably to refer to a pharmacologically active substance or composition. These terms of art are well-known in the pharmaceutical and medicinal arts.

As used herein, the terms “administration,” and “administering” refer to the manner in which a drug is presented to a subject. Administration can be accomplished by various routes well-known in the art such as oral, and non-oral methods.

As used herein, “transdermal” refers to the route of administration that facilitates transfer of a drug through a skin surface wherein a transdermal composition is administered to the skin surface.

As used herein, “skin,” “skin surface,” “derma,” and “epidermis” may be used interchangeably, and are meant to include not only the outer skin of a subject comprising one or more of epidermal layers, but also to include mucosal surfaces to which a drug composition may be administered. Examples of mucosal surfaces include the mucosa of the respiratory (including nasal and pulmonary), oral (mouth and buccal), vaginal, and rectal cavities. Hence the terms “transdermal” may encompass “transmucosal” as well.

As used herein, “enhancement,” “penetration enhancement,” or “permeation enhancement,” refer to an increase in the permeability of the skin, to a drug, so as to increase the rate at which the drug permeates through the skin. Thus, “permeation enhancer,” “penetration enhancer,” or simply “enhancer” refers to an agent, or mixture of agents that achieves such permeation enhancement. Several compounds have been investigated for use as penetration enhancers. See, for example, U.S. Pat. Nos. 5,601,839; 5,006,342; 4,973,468; 4,820,720; 4,006,218; 3,551,154; and 3,472,931. An index of permeation enhancers is disclosed by David W. Osborne and Jill J. Henke, in their publication entitled Skin Penetration Enhancers Cited in the Technical Literature, published in “Pharmaceutical Technology” (June 1998), which may also be found at the worldwide web address known as: pharmtech.com/technical/osborne/osborne.htm, which is incorporated by reference herein.

An “effective amount” of an enhancer refers to an amount sufficient to increase penetration of a drug through the skin, to a selected degree. Methods for assaying the characteristics of permeation enhancers are well-known in the art. See, for example, Merritt et al., Diffusion Apparatus for Skin Penetration, J. of Controlled Release 61 (1984), incorporated herein by reference in its entirety. By “effective amount” or “therapeutically effective amount,” or similar terms is meant a non-toxic but sufficient amount of a drug, to achieve therapeutic results in treating a condition for which the drug is known to be effective. The determination of an effective amount is well-within the ordinary skill in the art of pharmaceutical and medical sciences. See for example, Curtis L. Meinert & Susan Tonascia, Clinical Trials: Design, Conduct, and Analysis, Monographs in Epidemiology and Biostatistics, vol. 8 (1986).

As used herein, “pharmaceutically acceptable carrier,” and “carrier” may be used interchangeably, and refer to any inert and pharmaceutically acceptable material that has substantially no biological activity, and makes up a substantial part of the formulation. The carrier may be polymeric, such as an adhesive, or non-polymeric and is admixed with other components of the composition (e.g., drug, binders, fillers, penetration enhancers, anti-irritants, emollients, lubricants, etc., as needed) to comprise the formulation.

The term “admixed” means that the drug and/or enhancer can be dissolved, dispersed, or suspended in the carrier.

By the term “matrix”, “matrix system”, or “matrix patch” is meant a composition comprising an effective amount of a drug dissolved or dispersed in a polymeric phase, which may also contain other ingredients, such as a permeation enhancer diluents, skin irritation reducing agents, excipients, plasticizers, emollients, and other optional ingredients. This definition is meant to include embodiments wherein such polymeric phase is laminated to a pressure sensitive adhesive or used within an overlay adhesive.

A matrix system may also comprise an adhesive layer having an impermeable film backing attached onto the distal surface thereof and, before transdermal application, a release liner on the proximal surface of the adhesive. The film backing protects the polymeric phase of the matrix patch and prevents release of the drug and/or optional ingredients to the environment. The release liner functions similarly to the impermeable backing, but is removed from the matrix patch prior to application of the patch to the skin as defined above. Matrix patches with the above-described general characteristics are known in the art of transdermal delivery. See, for example, U.S. Pat. Nos. 5,985,317, 5,783,208, 5,626,866, 5,227,169, which are incorporated by reference.

As used herein, “liquid reservoir system,” its acronym “LRS,” or “liquid reservoir patch” refers to a transdermal delivery patch or system, in which a drug and other optional ingredients, such as a permeation enhancer, are admixed with a fluid carrier vehicle of desired viscosity, such as a gel or ointment, which is formulated for confinement in a reservoir having an impermeable backing and a skin contacting permeable membrane, or membrane adhesive laminate providing diffusional contact between the reservoir contents and the skin. For application, a peelable release liner is removed and the patch is attached to the skin surface. LRS patches are known in the art of transdermal drug delivery. Examples without limitation, of LRS transdermal patches are those described or referred to in U.S. Pat. Nos. 4,849,224, 4,983,395, which are incorporated by reference in their entirety.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a concentration range of 0.1 to 5 ng/ml should be interpreted to include not only the explicitly recited concentration limits of 0.1 ng/ml and 5 ng/ml, but also to include individual concentrations such as 0.2 ng/ml, 0.7 ng/ml, 1.0 ng/ml, 2.2 ng/ml, 3.6 ng/ml, 4.2 ng/ml, and sub-ranges such as 0.3-2.5 ng/ml, 1.8-3.2 ng/ml, 2.6-4.9 ng/ml, etc. This interpretation should apply regardless of the breadth of the range or the characteristic being described.

B. The Invention

As described above, the present invention provides transdermal compositions and methods for the delivery of female hormones, such as estrogens and progestins. It has been determined that norethindrone acetate does not convert to norethindrone in situ in the transdermal formulation, but rather, is only converted to norethindrone after it has migrated out of the formulation, and into the serum of a subject receiving the medication. Accordingly, Applicants have discovered that coadministration of norethindrone and norethindrone acetate to the skin of a subject can yield various advantages over transdermal administration of either norethindrone or norethindrone acetate alone. For example, in one aspect, transdermal co-delivery of norethindrone and norethindrone acetate may result in a synergistic effect that attains a higher norethindrone serum concentration in the subject, than is attained by delivering an equivalent amount of only norethindrone or only norethindrone acetate. In another aspect, transdermal coadministration of norethindrone and norethindrone acetate may provide a subject with a maximum norethindrone serum concentration within about 24 hours after initiation of administration. Such synergistic and unexpected results represent a significant advancement in the relevant art.

In one aspect, the female hormones to be delivered may be norethindrone and norethindrone acetate. In another aspect, the female hormones may further include an estrogenic hormone. A number of estrogenic hormones may be suitable for use in the transdermal compositions of the present invention, and specific hormones may be selected by one of ordinary skill in the art in order to attain a particularly desired result. Examples of estrogenic hormones that can be used in the present transdermal formulations include without limitation, 17-β-estradiol, 17-α-estradiol, estriol, estrone, and phytoestrogens. These estrogens may be derivatized or modified to form, for example, conjugated equine estrogens, esterified estrogens, ethinyl estradiol, etc. Examples of esterified estrogens include but are not limited to: estradiol-3,17-diacetate, estradiol-3-acetate, estradiol-17-acetate, estradiol-3,17-divalerate, estradiol-3-valerate, estradiol-17-valerate. However, in one aspect, the estrogenic hormone may be an estradiol. In another aspect, the estradiol may be ethinyl estradiol.

The specific amount of norethindrone and norethindrone acetate to be include in the transdermal formulations of the present invention may be a matter of choice depending on a specifically desired result to be achieved. However, in one aspect, the norethindrone amount may be from about 0.01% w/w to about 25% w/w of the formulation, and the norethindrone acetate amount may be from about 0.01% w/w to about 20% w/w. In a further aspect, the norethindrone amount may be from about 0.3% w/w to about 5% w/w of the formulation, and the norethindrone acetate amount may be from about 3% w/w to about 25% w/w of the formulation. In another aspect, the norethindrone amount may be from about 0.5% w/w to about 3% w/w, and the norethindrone acetate amount may be from about 3% w/w to about 12% w/w of the formulation. In yet another aspect, the norethindrone amount may be from about 1% w/w to about 2% w/w, and the norethindrone acetate amount may be from about 4% w/w to about 8% w/w of the formulation. In a further aspect, the norethindrone amount may be from about 1.5% w/w to about 2.5% w/w of the formulation. In an additional aspect, the norethindrone amount may be about 1% of the formulation and the norethindrone acetate amount may be about 7.5% of the formulation.

Alternatively, it is possible to quantify the amount of norethindrone with respect to the amount of norethindrone in a formulation as a ratio. In one aspect, the norethindrone and norethindrone acetate may be present in the formulation at a weight ratio of from about 1:1 to about 1:25. In another aspect, the ratio may be from about 1:2 to about 1:8. Other specific ratios may be selected by one of ordinary skill in the art in order to design a product with specifically desired characteristics.

Additionally, the specific amount of estrogenic hormone to be included in the transdermal compositions of the present invention may vary based on a number of criteria. The specific estrogen to be delivered, the other components included in the formulation, and the serum concentrations or profiles to be obtained, may all be taken into consideration. However, in one aspect, amount of estrogenic hormone may be sufficient to provide a therapeutic effect that is substantially equivalent to an effect produced by ethinyl estradiol administered from an adhesive matrix patch in an amount of from about to about 20 to 60 ug/cm². In another aspect, the amount of estrogenic hormone may be sufficient to provide a therapeutic effect that is substantially equivalent to an effect produced by ethinyl estradiol administered from an adhesive matrix patch in an amount of from about 25 to 45 ug/cm². In a further aspect, the amount of estrogenic hormone may be sufficient to provide a therapeutic effect that is substantially equivalent to an effect produced by ethinyl estradiol administered from an adhesive matrix patch in an amount of about 37.5 ug/cm². Those of ordinary skill in the art will recognize a number of routine mechanisms for determining an estrogenic hormone amount that is sufficient to provide a therapeutic effect equivalent to a specified ethinyl estradiol concentration, and that such determinations may be readily made without undue experimentation.

In addition to the desired amount and number of bioactive agents, the transdermal formulations of the present invention may also optionally include a permeation enhancer, or mixture of permeation enhancers. As more fully enumerated in the examples below, it has been found that lauryl-type and polyol-type agents provide a significant penetration enhancing effect on norethindrone and norethindrone acetate.

The specific lauryl or polyol-type enhancer, and the amount thereof, may be selected by one of ordinary skill in the art depending on a specific result to be achieved. However, as a general matter, the amount of enhancer included in the transdermal formulation may be from about 0.01% w/w to about 50% w/w of the formulation. In a more detailed aspect, the amount of enhancer may be from about 3% w/w to about 16% w/w of the formulation. In a further aspect, the amount of enhancer may be about 8% w/w of the formulation. In an additional aspect, the amount of enhancer may be about 5% w/w of the formulation.

In one aspect, the enhancer included in the formulation may be a lauryl-type enhancer. A variety of lauryl-type enhancers may be suitable for use in the present invention. However, in one aspect the lauryl-type enhancer used may include without limitation, lauryl alcohol, 1-lauryl-2-pyrrolidone, and mixtures thereof. In another aspect, the enhancer may be a mixture of lauryl alcohol in an amount of about 5% w/w and 1-lauryl-2-pyrrolidone in an amount of about 3% w/w. In yet another aspect, the enhancer may be a polyol-type enhancer. Further, a variety of polyol-type enhancers may be suitable for use in the present invention. However, in one aspect, the polyol-type enhancer used may be dipropylene glycol.

The transdermal formulation of the present invention may take the form of an occlusive device, such as a transdermal patch. Such a transdermal patch may either be an adhesive matrix patch, a liquid reservoir system type patch, a buccal tablet, or the like. A wide variety of each specific device type are known to those of ordinary skill in the art. Optional ingredients such as adhesives, excipients, backing films, release liners, etc., and the required amount of each will vary greatly depending upon the type of patch desired, and may be determined as needed by one ordinarily skilled in the art to arrive at a specific formulation with desired characteristics and properties.

In one aspect of the present invention the formulation may be a transdermal adhesive matrix patch. In some aspects, such matrix patches may include a backing member, a polymeric adhesive matrix, and the active agents. A removable protective release liner may be provided to protect the drug-containing adhesive matrix until ready for use. Monolithic systems where the drug is contained directly in a single pressure sensitive adhesive layer, as well as systems containing one or more polymeric reservoirs in addition to the pressure sensitive adhesive layer may be used. As noted above, in one aspect of the present invention, a permeation enhancer may be used to increase the delivery rate of the drug, and may also be used to vary other parameters, such as patch size, etc.

Polymeric adhesives suitable for use in the present invention may include, but are not limited to, crosslinked or uncrosslinked acrylic copolymers (e.g. DUROTAK 2516, 2074, etc. National Starch and Chemical Co.), acrylics, vinyl acetates, natural and synthetic rubbers, ethylenevinylacetate copolymers, polysiloxanes, polyacrylates, polyurethanes, polyisobutylene copolymers, polyether block amide copolymers, and mixtures thereof. Those of ordinary skill in the art will appreciate that the specific type and amount of adhesive polymer used may be selected depending upon the desired specific characteristics of the final product. However, in one aspect, the amount of adhesive polymer in the adhesive matrix layer may be at least about 50% w/w of the adhesive layer. In another aspect, the amount may be at least about 60% w/w of the adhesive layer. In yet another aspect, the amount may be at least about 85% w/w of the adhesive layer. In a further aspect, the amount may be at least about 90% w/w of the adhesive layer. In an additional aspect, the amount may be from about 50% w/w to about 95% w/w of the adhesive layer.

As noted above, in accordance with the present invention, the drug-containing adhesive matrix layer can, in addition to the polymeric adhesive matrix and drug, also contain other optional ingredients, such as carriers, vehicles, permeation enhancers, excipients, diluents, emollients such as glycerin, and the like, which are suitable for administration in conjunction with the present invention. Such materials are pharmaceutically acceptable in that they are nontoxic, do not hinder drug delivery, and are not for any other reasons biologically or otherwise undesirable. Examples of such additional materials include water, mineral oil, silicone, inorganic gels, aqueous emulsions, liquid sugars, waxes, petroleum jelly, and a variety of other oils and polymeric materials. Those of ordinary skill in the art will be able to select specific additives in specific amounts in order to provide a matrix patch with particularly desired characteristics.

In another aspect of the present invention, polymers useful for the backing layer are polyethylene, polypropylene, polyesters, polyurethanes, polyethylene vinyl acetate, polyvinylidene chloride, block copolymers such as PEBAX, and the like.

The formulations of the present invention include sustained release formulations that administer therapeutically effective amounts of norethindrone and norethindrone over an extended period of time. However, in one aspect, a sustained delivery the sustained delivery period of norethindrone and norethindrone acetate may be for at least 7 days. In another aspect, the sustained delivery period may be at least 5 days. In a further aspect, the sustained delivery period may be at least 3 days.

In addition to the transdermal formulations and compositions disclosed herein, the present invention includes methods for the making and use thereof. In one aspect, a norethindrone serum concentration in a subject that was achieved by transdermal delivery of either norethindrone alone or norethindrone acetate alone may be exceeded by transdermally administering an equivalent combined amount of norethindrone and norethindrone acetate. In another aspect, a maximum norethindrdne serum concentration may be achieved in a subject within about 24 hours after initiation of transdermal administration by delivering a combination of norethindrone and norethindrone acetate. In yet another aspect, norethindrone and norethindrone acetate permeation through the skin of the subject may be enhanced by utilizing the enhancers enumerated herein.

### EXAMPLES

The following examples of norethindrone formulations are provided to promote a more clear understanding of certain embodiments of the present invention, and are in no way meant as a limitation thereon.

### Example 1

Transdermal matrix systems containing norethindrone and norethindrone acetate were made as follows. The solids contents of an acrylic adhesive solution, (Durotak 87-2074) was determined by placing small amounts into pre-weighed aluminum dishes which were then put in a convection oven (Model A4718-Q, Blue M) at 75° C. overnight. Following evaporation of the solvents, the weight of the dry adhesive was obtained and the solids content calculated as a ratio of the dry to wet weight.

The adhesive 87-2074 contains approximately 28-31% solids and was always used undiluted. Known quantities of the adhesive were weighed into glass bottles based on the previously determined solids content. For all the formulations, appropriate quantities of norethindrone (NE) were first added to the liquid adhesive in each bottle (to give 1% w/w drug content upon drying). The bottles were capped and sealed with parafilm and rotated until all the NE was dissolved. Appropriate quantities of norethindrone acetate (NEA) (to give 7.5% w/w drug upon drying) were added to the bottle of the formulation in which no enhancer is desired. To the other bottles, appropriate quantities of norethindrone acetate and lauryl alcohol or 1-lauryl-2-pyrrolidone (LP-300) or mixtures of 1 auryl alcohol and 1-lauryl-2-pyrrolidone (LP-300) were added to the bottles containing norethindrone in adhesive to give the desired compositions upon drying. Each bottle was again tightly capped, sealed with parafilm and rotated overnight during which time the NE or NEA and enhancers had dissolved to yield a clear solution.

A small amount of each formulation (about 10 g) was placed onto the high release side of a silicone release-coated 3 Mil thick polyester (PET) liner (Loparex Inc., 10393S) and manually cast with a 10 Mil gap casting knife. Each cast was placed in a convection oven (Model A4718-Q, Blue M) at 75° C. for 15 minutes. After drying, each cast was laminated with a 3 Mil polyethylene (PET) monolayer backing film (3M, CoTran™ 9720).

### Example 2

Utilizing adhesive matrix patches made in accordance with the above-recited procedure, in vitro skin flux studies were conducted using modified Franz diffusion cells. Heat separated human cadaver epidermal membranes were used. The matrix patches for each formulation were cut into 0.71 cm² circular discs. The release liner was peeled and discarded and the matrix disc laminated onto the stratum corneum side of the epidermal membrane. The skin-matrix assembly was then sandwiched between the donor and receiver chambers of a diffusion cell and clamped in place with the epidermal side facing the receiver compartment. The receiver compartment was filled with 0.02% w/v sodium azide (NaN₃) solution. The cells were then placed in a circulating water bath maintained at 32±1° C.

At time points of 24, 48, 72, 96, 120, 144 and 168 hrs, the entire contents of the receiver compartment were collected for drug quantitation. The receiver compartment was then re-filled with fresh receiver medium. The interval flux and cumulative amount of drug permeating per unit area were calculated following HPLC analyses of the samples. The flux study results are contained in Tables 1 and 2 below.

TABLE 1
A comparison of transdermal matrix formulations with and without
lauryl-type enhancers
DAILY DELIVERY (μg/cm²)

| | 5% LA | | |
|---|---|---|---|
| NO | (Mean ± | 3% LP-300 | 5% LA, 3% LP-300 |
| ENHANCER | sd)** | (Mean ± sd)* | (Mean ± sd)* |
| (Mean ± sd)* | 2074/NEA/ | 2074/NEA/NE/ | 2074/NEA/NE/ |
| 2074/NEA/NE | NE/LA | LP-300 | LA/LP-300 |
| 91.5/7.5/1 | 86.5/7.5/1/5 | 88.5/7.5/1/3 | 83.5/7.5/1/5/3 |
| 10.9 ± 3.1 | 23.9 ± 6.1 | 25.7 ± 9.6 (236%) | 29.7 ± 8.5 (272%) |
| | (219%) | | |
| 10.6 ± 3.0 | 20.3 ± 4.7 | 19.8 ± 6.2 (187%) | 22.7 ± 6.8 (214%) |
| | (192%) | | |
| 9.6 ± 2.4 | 17.2 ± 4.4 | 16.8 ± 5.2 (175%) | 18.4 ± 5.0 (192%) |
| | (179%) | | |
| 8.7 ± 2.0 | 16.4 ± 3.0 | 16.4 ± 4.7 (189%) | 18.0 ± 4.5 (207%) |
| | (189%) | | |
| 7.7 ± 2.1 | 16.2 ± 3.6 | 16.7 ± 5.0 (217%) | 17.6 ± 3.8 (229%) |
| | (210%) | | |
| 7.3 ± 1.6 | 15.1 ± 3.3 | 15.2 ± 4.6 (208%) | 15.9 ± 3.2 (218%) |
| | (207%) | | |
| 7.6 ± 1.4 | 14.2 ± 3.1 | 14.6 ± 3.7 (192%) | 14.7 ± 2.8 (193%) |
| | (187%) | | |
| Average | 198 | 205 | 218 |
| Enhancement | | | |

*n = 6
**n = 3

TABLE 2
A comparison of transdermal formulations with and without
polyoltype enhancers
Daily Delivery (ug/cm²)

| | NO ENHANCER (Mean ± sd)* | 8% DPG (Mean ± sd)* |
|---|---|---|
| DAY | 2074/NEA/NE 91.5/7.5/1 | 2074/NEA/NE/DPG 83.5/7.5/1/8 |
| DAY | 10.9 ± 3.1 | 22.4 ± 8.2 (206%) |
| 1 | | |
| DAY | 10.6 ± 3.0 | 16.0 ± 6.1 (151%) |
| 2 | | |
| DAY | 9.6 ± 2.4 | 13.1 ± 4.7 (136%) |
| 3 | | |
| DAY | 8.7 ± 2.0 | 12.8 ± 3.7 (147%) |
| 4 | | |
| DAY | 7.7 + 2.1 | 12.9 ± 3.8 (168%) |
| 5 | | |
| DAY | 7.3 + 1.6 | 11.2 ± 2.9 (153%) |
| 6 | | |
| DAY | 7.6 + 1.4 | 11.0 ± 2.6 (145%) |
| 7 | | |
| | Average enhancement | 158 |

*n = 3

As can be seen from the above-recited results, each of the enhancers tested showed significant increases in penetration as compared to formulations containing no enhancer. Further, as can be seen by both the formulations with and without enhancers, the combination of NE and NEA in all cases produced peak flux rates within the first 24 hours following initiation of administration, with flux rate gradually declining over the sustained release period of 7 days. As such, it can be concluded that the combination of NE and NEA will provide maximum norethindrone serum concentrations within about 24 hours of administration initiation, and will further provide good plasma concentrations over a sustained release period of at least 7 days.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements.

Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A transdermal composition for administration to a subject comprising:
a pharmaceutically acceptable transdermal carrier; and
a therapeutically effective amount of norethindrone and norethindrone acetate in the carrier.

2. The transdermal composition of claim 1, wherein the norethindrone and norethindrone acetate are present in a weight ratio of from about 1:1 to about 1:25.

3. The transdermal composition of claim 2, wherein the ratio of norethindrone to norethindrone acetate is from about 1:2 to about 1:8.

4. The transdermal composition of claim 1, wherein the composition provides a maximum norethindrone serum concentration in the subject within about 24 hours after initiation of administration.

5. The transdermal composition of claim 1, wherein the composition, when administered, provides the subject with a substantially higher norethindrone serum concentration than an equivalent dosage of a transdermal composition containing either norethindrone or norethindrone acetate alone.

6. The transdermal composition of claim 1, further comprising a therapeutically effective amount of an estrogenic hormone.

7. The transdermal composition of claim 6, wherein said estrogenic hormone is a an estradiol.

8. The transdermal composition of claim 7, wherein the estradiol is ethinyl estradiol.

9. The transdermal composition of claim 6, wherein the amount of estrogenic hormone is sufficient to provide a therapeutic effect that is substantially equivalent to an effect produced by ethinyl estradiol administered from an adhesive matrix patch in an amount of from about to about 25 to 45 ug/cm².

10. The transdermal composition of claim 1, further comprising an effective amount of a penetration enhancer selected from the group consisting of: lauryl-type enhancers, polyol-type enhancers, and mixtures thereof.

11. The transdermal composition of claim 10, wherein the penetration enhancer is a lauryl-type enhancer selected from the group consisting of: lauryl alcohol, 1-lauryl-2-pyrrolidone, and mixtures thereof.

12. The transdermal composition of claim 11, wherein the lauryl-type enhancer is a mixture of lauryl alcohol and 1-lauryl-2-pyrrolidone.

13. The transdermal composition of claim 12, wherein the penetration enhancer is a polyol-type enhancer.

14. The transdermal composition of claim 13, wherein the polyol-type enhancer is dipropylene glycerol.

15. The transdermal composition of claim 10, wherein the enhancer amount is from about 3% w/w to about 8% w/w of the transdermal composition.

16. The transdermal composition of claim 1, wherein the carrier comprises a polymeric adhesive matrix.

17. The transdermal composition of claim 16, wherein the polymeric adhesive layer comprises an acrylic pressure-sensitive adhesive.

18. A method of transdermally providing a subject with a maximum norethindrone serum concentration within about 24 hours after initiation of transdermal administration comprising:
transdermally coadministering norethindrone and norethindrone acetate to the skin of the subject.

19. A method of exceeding a norethindrone serum concentration achieved in a subject by transdermal delivery of either norethindrone alone or norethindrone acetate alone, comprising:
administering to the skin of a subject a combined amount of norethindrone and norethindrone acetate that is equivalent to an amount of either the norethindrone or norethindrone acetate alone.

20. A method of enhancing norethindrone and norethindrone acetate permeation through the skin of a subject comprising:
coadministering the norethindrone and norethindrone acetate with a permeation enhancer selected from the group consisting of: lauryl alcohol, 1-lauryl-2-pyrrolidone, dipropylene glycerol, and mixtures thereof to the skin.
